SEQUENCE LISTING (10 sequences)

SEQ ID NO: 1 - DNA, 25 bp, Artificial Sequence
  primer
gatgtatctatctggaactctagtg

SEQ ID NO: 2 - DNA, 32 bp, Artificial Sequence
  primer
tggtaaatcatgtaaatagtaataatcaagcc

SEQ ID NO: 3 - DNA, 1467 bp, Streptococcus agalactiae
atattggtaagaagaaatttccttaaaaataagattaaataggttgtaaagtatccgtatgggttttacttgaaaaactaaattaaattatcaagaaattaccccccaggataggcgccaagaatattatacccacttgataatggtaagttttatgctaaaaatgcagtttacttgtaataatgttaaatatagggggaaagaaagcgctttgacgaccttttggacaagtagtaagataccaacatgggccctgtaaattaaaaatactgcagtagaagtgattttagtttaaaggaggaaatttattatgaacgttacacatatgatgtatctatctggaactctagtggctggtgcattgttattttcaccagctgtattagaagtacatgctgatcaagtgacaactccacaagtggtaaatcatgtaaatagtaataatcaagcccagcaaatggctcaaaagcttgatcaagatagcattcagttgagaaatatcaaagataatgttcagggaacagattatgaaaaaccggttaatgaggctattactagcgtggaaaaattaaagacttcattgcgtgccaaccctgagacagtttatgatttgaattctattggtagtcgtgtagaagccttaacagatgtgattgaagcaatcactttttcaactcaacatttaacaaataaggttagtcaagcaaatattgatatgggatttgggataactaagctagttattcgcattttagatccatttgcttcagttgattcaattaaagctcaagttaacgatgtaaaggcattagaacaaaaagttttaacttatcctgatttaaaaccaactgatagagctaccatctatacaaaatcaaaacttgataaggaaatctggaatacacgctttactagagataaaaaagtacttaacgtcaaagaatttaaagtttacaatactttaaataaagcaatcacacatgctgttggagttcagttgaatccaaatgttacggtacaacaagttgatcaagagattgtaacattacaagcagcacttcaaacagcattaaaataatatttgtatttttcgtgtgatgctgtcgacttcgtgattttgtactaccatgattgttatgattaaaagatttacgacaatagtcataatagtagaacgatgtcaccattttaaataataaagtgattagtcatttgactaaatttgccaagtatcaaaggaaataaagattatgactaaaaagataactgttgtagcattagaaacattgattgcccagcataataatatccatttgatagacgttcgtgaagagcatgagtatcgtggagggcatattccaggtgcgataaatcttcctttgagtcactcagtcataagtttgaacagttagataaaataaggaatattatcttgttggcaacgagggggaagatctattagagcat

SEQ ID NO: 4 - DNA, 124 bp, Streptococcus agalactiae
gatgtatctatctggaactctagtggctggtgcattgttattttcaccagctgtattagaagtacatgctgatcaagtgacaactccacaagtggtaaatcatgtaaatagtaataatcaagcc

SEQ ID NO: 5 - DNA, 33 bp, Artificial Sequence
  probe
agaagtacatgctgatcaagtgacaactccaca

SEQ ID NO: 6 - DNA, 21 bp, Artificial Sequence
  primer
gtgcatcaccagagcatgtat

SEQ ID NO: 7 - DNA, 26 bp, Artificial Sequence
  primer
cagctacagacagtaagttacaagcg

SEQ ID NO: 8 - DNA, 1301 bp, Streptococcus agalactiae
atgaaaatgaataaaaaggtactattgacatcgacaatggcagcttcgctattatcagtcgcaagtgttcaagcacaagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaataacattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaataa

SEQ ID NO: 9 - DNA, 80 bp, Streptococcus agalactiae
gtgcatcaccagagcatgtatcagctccagcagttcctgtgactacgacttcaacagctacagacagtaagttacaagcg

SEQ ID NO: 10 - DNA, 29 bp, Artificial Sequence
  probe
agctccagcagttcctgtgactacgactt

(19) United States
(12) Patent Application Publication
(10) Pub. No.: US 2003/0207273 A1
(43) Pub. Date: Nov. 6, 2003

(54) Polynucleotide primers and probes for rapid detection of Group B streptococcal (GBS)

(76) Inventors: Betty Wu, Canton, MI (US); Purnima Kurnool, Canton, MI (US)

Correspondence Address: KLAUBER & JACKSON, 411 HACKENSACK AVENUE, HACKENSACK, NJ, 07601

(21) Appl. No.: 10/102,513

(22) Filed: Mar. 20, 2002

(51) Int. Cl. C12Q 1/68; C12P 19/34

(52) U.S. Cl. 435/6; 435/91.2

(57) ABSTRACT

Highly specific oligonucleotide primers and probes useful in a rapid and specific method for detecting the presence of Group B Streptococcal (GBS) or Streptococcus agalactiae infection in a biological sample.

### FIELD OF THE INVENTION

This invention relates to methods for detecting Group B streptococci (GBS) infections, particularly to methods allowing a rapid and accurate diagnosis to prevent and treat neonatal GBS infections.

### BACKGROUND

Group B streptococci (GBS) are responsible for a broad range of severe human diseases, predominantly the life-threatening bacterial infections in neonates and very young infants. Approximately 70 to 80% of infant infections occur in the first few days of life, so-called early-onset disease, while late-onset infections occur in infants between 1 week and 3 months of age. Newborns with early-onset GBS disease usually acquire the organism during delivery from their GBS-colonized mothers, manifesting in sepsis and meningitis which cause not only illness and death, but long term disabilities such as hearing loss, impaired vision, developmental problems, and cerebral palsy.

In order to substantially reduce the incidence of early-onset GBS disease, prenatal screening for GBS and intrapartum antimicrobial prophylaxis are now highly recommended in the United States. However, since these strategies require the frequent use of antibiotics, antibiotic resistant GBS or other bacterial agents might emerge during the perinatal period. In addition, these measures are unlikely to prevent late-onset infections, prematurity, and stillbirths related to GBS, while obviously not addressing GBS disease in nonpregnant adults. GBS are increasingly recognized as a frequent cause of invasive infections in pregnant women and clinically ill and older adults, such as those suffering from diabetes, cirrhosis, malignancies and immunodifciencies.

Currently, culture, including broth culture in selective medium, is the gold standard method for detection of GBS. However, the culture methods require up to 36 hours to obtain results and predict only 87% of women likely to be colonized by GBS at delivery. A rapid, sensitive, and specific test for detection of GBS directly from clinical specimens would allow for a simpler and more efficient prevention program.

Rapid tests have been developed, such as the rapid antigen-based tests, but these tests are neither sensitive nor specific enough to substitute for bacterial culture. The most widely used hybridization-based test to date is the Accuprobe Group B Streptococcocus Identification Test™ (Gen-Probe, San Diego, Calif.). GBS-specific polymerase chain reaction (PCR) assays using real-time PCR have been described which provide improved detection of GBS (Bergeron et al. (2000) N. Engl. J. Med. 343, 175-179).

### BRIEF SUMMARY OF THE INVENTION

The present invention features a rapid and accurate PCR-based assay for Streptococcus agalactiae, the organism responsible for neonatal Group B Streptococcal (GBS) infections.

The method of the invention presented here included a pair of hybridization primers (SEQ ID NO: 1 and 2) specific to the portion of the cfb gene (FIG. 1; SEQ ID NO: 3) between positions 328 and 451 (SEQ ID NO:4) encoding the CAMP factor. The CAMP factor is a diffusible extracellular protein and is produced by the majority of GBS. The gene encoding CAMP factor, cfb gene (GenBank access number: X72754), is present in virtually every GBS isolate and has been used for the development of a PCR based identification of GBS (Danbing K. et al., 2000, Clinical Chemistry, 46, 324-331).

Further, the instant invention also provides a specific probe (SEQ ID NO: 5) designed to recognize the sequence amplified between the primers, e.g., the amplicons of the cfb gene comprised of the 123 bp sequence of SEQ ID NO:4, allowing real-time detection by using fluorescence measurements.

The present invention also includes a pair of GBS specific PCR amplification primers (SEQ ID NO: 6 and 7) specific for a portion of the sip gene (FIG. 2; SEQ ID NO:8) between positions 778 and 857 (SEQ ID NO:9). GBS sip gene (GenBank access number: AF151357, AF151358, AF151359, AF151360, AF151361, AF151362) encodes a 53-kDa protein called surface immunogenic protein (“Sip”), which is present in all serotypes of GBS. Further included is a specific probe (SEQ ID NO: 10) recognizing the amplicons allowing real-time detection by using fluorescence measurement.

Accordingly, in a first aspect, the invention features a method of testing for the presence of Streptococcus agalactiae, comprising:

(a) hybridization a nucleic acid sample suspected of being infected with Group B Streptococcal (GBS), with (i) a first pair of CAMP-based Group B Streptococcal (GBS)-specific primers, and (ii) a second pair of Sip-based GBS-specific primers, under conditions wherein GBS-related nucleic acids are amplified; and

(b) detecting the presence of GBS-related nucleic acids.

In one embodiment, the first pair of CAMP-based GBS primers are the oligonucleotides of SEQ ID NOs:1 and 2. In another embodiment, the second pair of Sip-based GBS primers are the oligonucleotides of SEQ ID NOs:5 and 6. In a further embodiment, the first pair of CAMP-based GBS primers are the oligonucleotides of SEQ ID NOs:1 and 2 and the second pair of Sip-based GBS primers are the oligonucleotides of SEQ ID NOs:5 and 6.

In one embodiment, the method of the invention is used in conjunction with SYGR as a means of amplicon detection. SYGR is a fluorescent dye which binds to double stranded DNA and fluoresces strongly when bound to double stranded DNA. In another embodiment, step (a) is conducted in the presence of the probe of SEQ ID NOs:5. In another embodiment, step (a) is conducted in the presence of the probe of SEQ ID NO:10. In a more specific embodiment, step (a) is conducted in the presence of the pro be of SEQ ID NOs:5. In another embodiment, step (a) is conducted in the presence of the probes of SEQ ID NOs:5 and 10.

The probes of SEQ ID NOs: 5 and 10 are double labeled with a fluorophore at the 5′ and a quencher at the 3′, so when the probe is intact the flourophore is not able to fluorescence (TaqMan®Probe, IDT, Coralville, Iowa). During PCR extension, the probes hybridized to the amplicons is cleaved by the 5′-3′ exonuclease activity of Taq polymerase, resulting releasing of the 5′ fluorophore from its quencher. The intensity of the fluorescence increases as more amplicons being synthesized.

In a further embodiment, the nucleic acid sample is a biological sample obtained from a patient to be tested for the presence of GBS. DNA from any biological sample extracted by any known method may be used in the method of the invention. Biological samples include, for example, vaginal or anal specimens, amniotic fluid, spinal fluid, or plasma.

In a further embodiment, step (a) in conducted in a volume of 0.2-100 μl; in further embodiments, step the reaction is conducted in a volume of less than 50 μl, or less than 25 μl; in a still further embodiment, the reaction is conducted in less than 15 μl.

In a second aspect, the invention features a method of diagnosing a Group B Streptococcal (GBS) infection, comprising:

(a) contacting a GBS-related target nucleic acid with (i) a first pair of CAMP-based Group B Streptococcal (GBS)-specific primers, (ii) a CAMP-based Group B Streptococcal (GBS)-specific probe, (iii) a second pair of Sip-based GBS-specific primers, and (iv) a Sip-based GBS-specific probe, under conditions wherein GBS-related nucleic acids are amplified; and

(b) detecting the amplified products, wherein detection of amplified products indicates the presence of a GBS infection.

In a third aspect, the invention features an in vitro method for detecting the presence of Streptococcus agalactiae in a biological sample, comprising:

(a) releasing nucleic acids from said biological sample;

(b) performing PCR in a total volume of between 0.2-100 μl in the presence of a first pair of primers comprising SEQ ID NOs: 1 and 2, and a second pair of primers comprising SEQ ID NOs:6 and 7, and labeled probes comprising SEQ ID NOs:5 and 10, under conditions wherein the presence of a Streptococcus agalactiae-related nucleic acid sequence results in an amplified and labeled PCR product; and

(c) detecting the presence of PCR product with either specific probes or SYBG.

In a fourth aspect, the invention features a method detecting a Group B Streptococcal (GBS) infection in a patient, comprising:

(a) obtaining a biological sample from the patient;

(b) releasing nucleic acids from said biological sample;

(c) performing PCR in a total volume of between 0.2-100 μl in the presence of a first pair of primers comprising SEQ ID NOs:1 and 2, and a second pair of primers comprising SEQ ID NOs:6 and 7, and labeled probes comprising SEQ ID NOs:5 and 10, under conditions wherein the presence of a Streptococcus agalactiae-related nucleic acid sequence results in an amplified and labeled PCR product; and

(d) detecting the presence of PCR product with specific fluorescent labeled probes or SYBG, wherein the presence of degraded probe indicates the presence of a GBS infection.

Other objects and advantages will become apparent from a review of the ensuing detailed description taken in conjunction with the following illustrative drawing.

### BRIEF DESCRIPTION OF THE DRAWINGS

FIG. 1 is the cfb gene (SEQ ID NO: 3) showing primers (SEQ ID NO: 1 and 2) specific to the portion of the cJb gene between positions 328 and 451 (SEQ ID NO:4) encoding the CAMP factor, and a specific probe (SEQ ID NO: 5) designed to recognize the sequence amplified between the primers comprised of the 123 bp sequence of SEQ ID NO:4.

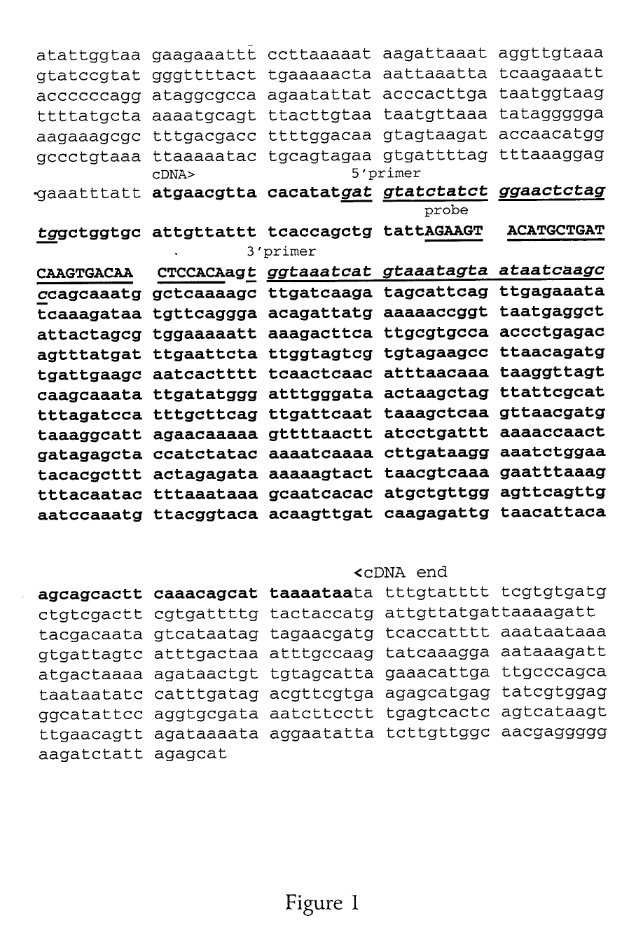

FIG. 2 is the sip gene (SEQ ID NO:8) with the positions of the primers (SEQ ID NO: 6 and 7) specific for a portion of the sip gene between positions 778 and 857 (SEQ ID NO:9) and a specific probe (SEQ ID NO: 10) recognizing the amplicons.

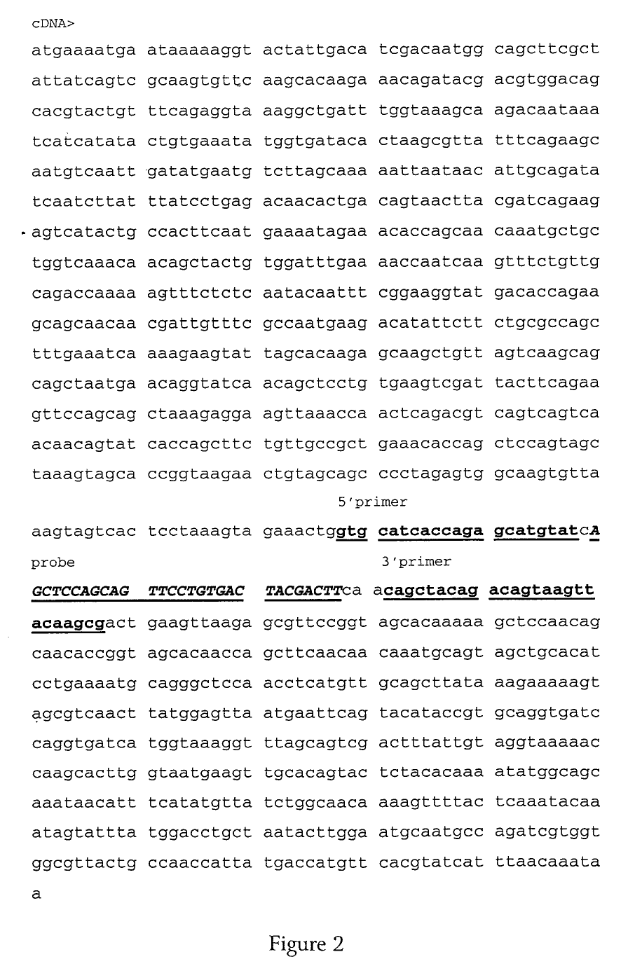

### DETAILED DESCRIPTION

Before the present methods and compositions are described, it is to be understood that this invention is not limited to particular methods, compositions, and experimental conditions described, as such methods and compounds may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

### DESCRIPTION OF THE INVENTION

The method described herein is a rapid and accurate screening test for GBS that can be performed at the time of delivery and which obviates the need for prenatal screening and reduce the use of antibiotic prophylaxis in women who are not colonized.

Recently, Bergeron et al. (2000) N. Engl. J. Med. 343, 175-179, described a rapid PCR technique capable of correctly identifying more than 90% women colonized with GBS. Both the sensitivity and specificity of this technique appeared to be in the range that would be acceptable for clinical use.

The method described in the instant specification uses a standard PCR machine Further, the probes and primers of the instant invention provide a very high and specific sensitivity for rapid detection of GBS.

Accordingly, the present invention resides in part in a process for amplifying two specific nucleic acid sequences present in a nucleic acid or mixture thereof, using two pairs of specific primers for polymerization, and two specific probes for detecting the amplified sequences. Further, the present invention provides an important advantage in allowing quick detection of the presence of the GBS pathogen so that appropriate medical intervention is available to the infection patient(s) more quickly.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Primer and Probe Design

The sequences of cfb and sip genes are obtained from GenBank. The primers and probes were designed with the aid of Primer Express 1.0 (PE Applied Biosystem). The possible homologies of the primers and probes with other none GBS genes were checked using NCBI Blast program and Megaline (DNA Star Lasergene).

Camp based GBS-specific primers:

| | | |
|---|---|---|
| Forward primer: | 5′ GATGTATCTATCTGGAACTCTAGTG 3′; | (SEQ ID NO:1) |
| Reverse primer: | 5′ GGCTTGATTATTACTATTTACATGATTTACCA 3′; | (SEQ ID NO:2) |
| Probe: | 5′ F-AGAAGTACATGCTGATCAAGTGACAACTCCACA-Q 3′. | (SEQ ID NO:5) |

Sip Based GBS-Specific Primers:

| | |
|---|---|
| Forward primer: | (SEQ ID NO:6) |

5′ GTGCATCACCAGAGCATGTAT 3′;

| | |
|---|---|
| Reverse primer″ | (SEQ ID NO:7) |

5′ CGCTTGTAACTTACTGTCTGTAGCTG 3′;

| | |
|---|---|
| Probe: | (SEQ ID NO:10) |

5′ F-AGCTCCAGCAGTTCCTGTGACTACGACTT 3′.

The specificity of the primers and probes was tested with real-time PCR (Taqman assay) using genomic DNAs isolated from the following organisms (listed in Table 1): nine GBS serotypes (serotype Ia, Ib, Ic, II, III, IV, V, VI and VII; American Type Culture Collection and National Center for Streptococcus, Canada); 10 clinical GBS isolates; 60 clinical samples; a wide variety of gram-positive and gram-negative bacterial strains as well as two yeast strains and HSV type 1 and 2.

TABLE 1

| PATHOGEN | TYPE |
|---|---|
| Pseudomonas aeruginosa | Gram − Bacteria |
| Proteus mirabilis | Gram − Bacteria |
| Klebsiella oxytoca | Gram − Bacteria |
| Klebsiella pneumoniae | Gram − Bacteria |
| Escherichia coli (clinical isolate 1) | Gram − Bacteria |
| Escherichia coli (clinical isolate 2) | Gram − Bacteria |
| Acinetobacter baumannii | Gram − Bacteria |
| Serratia marcescens | Gram − Bacteria |
| Enterobacter aerogenes | Gram + Bacteria |
| Enterococcus faecium | Gram + Bacteria |
| Staphylococcus aureus (clinical isolate 1) | Gram + Bacteria |
| Staphylococcus aureus (clinical isolate 2) | Gram + Bacteria |
| Streptococcus pyo genes | Gram + Bacteria |
| Streptococcus viridans | Gram + Bacteria |
| Listeria monocytogenes | Gram + Bacteria |
| Enterococcus sps. | Gram + Bacteria |
| Candida glabrata | Yeast |
| Candida albicans | Yeast |
| Streptococcus Group C | Gram + Bacteria |
| Streptococcus Group G | Gram + Bacteria |
| Streptococcus Group F | Gram + Bacteria |
| Enterococcus faecalis | Gram + Bacteria |
| Streptococcus pneumoniae | Gram + Bacteria |
| Staphylococcus epidermidis (C−) | Gram + Bacteria |
| Gardenerella vaginalis | Gram + Bacteria |
| Micrococcus sps. | Gram + Bacteria |
| Haemophilus influenzae | Gram − Bacteria |
| Neisseria gonorrhoeae | Gram − Bacteria |
| Moraxella catarrahlis | Gram − Bacteria |
| Salmonella sps. | Gram − Bacteria |
| Chlamydia trachomatis | Gram − Bacteria |
| Peptostreptococcus productus | Gram + Bacteria |
| Peptostreptococcus anaerobius | Gram + Bacteria |
| Lactobacillus fermentum | Gram + Bacteria |
| Eubacterium lentum | Gram + Bacteria |
| Herpes Simplex Virus I (HSV I) | Virus |
| Herpes Simplex Virus II (HSV II) | Virus |

Assay procedure. A typical PCR was conducted with the GBS specific probes and primers of the invention under the following conditions:

20 mM Tris-HCl, pH 8.4

50 mM KCl

4 mM MgCl2

0.2 mM dNTPs

400 μM primers (SEQ ID NOs:1, 2, 4, 5)

200 μM probes (SEQ ID NOs:3, 6)

10 fg to 1 ng DNA

1-2 U of Taq polymerase

Total volume is 15 μl and the reaction is carried out in a LightCycler with: 25 sec denaturing at 94° C.; followed by 50 cycles of 94° C. for 3 sec., and 60° C. for 20 sec.

Results. Both sets of primers and probes recognized all the nine GBS serotypes, the 10 clinical isolates, and the clinical samples, which are GBS positive by culturing method. There are no cross reactivities with any of the other pathogens.

We claim:

1. A method of testing for the presence of Streptococcus agalactiae, comprising:
(a) contacting a nucleic acid sample suspected of being infected with Group B Streptococcal (GBS), with (i) a first pair of CAMP-based Group B Streptococcal (GBS)-specific primers, and (ii) a second pair of Sip-based GBS-specific primers, under conditions wherein GBS-related nucleic acids are amplified; and
(b) detecting the presence of GBS-related nucleic acids.

2. The method of claim 1, wherein the first pair of CAMP-based Group B Streptococcal (GBS)-specific primers are the nucleic acid sequences of SEQ ID NOs:1 and 2.

3. The method of claim 1, wherein the second pair of Sip-based GBS-specific primers are the nucleic acid sequences of SEQ ID NOs:4 and 5.

4. The method of claim 1, wherein step (a) is conducted in the presence of labeled probes comprising SEQ ID NOs:3 and 6.

5. The method of claim 1, wherein the nucleic acid sample is a biological sample obtained from a patient to be tested for the presence of GBS.

6. The method of claim 1, wherein step (a) in conducted in a volume of between 0.2-100 μl.

7. The method of claim 1, wherein the nucleic acid is extracted from a biological sample obtained from a patient suspected of being infected with GBS.

8. A method of amplifying a nucleic acid related to Group B Streptococcal (GBS), comprising:
(a) contacting a GB S-related target nucleic acid with (i) a first pair of CAMP-based Group B Streptococcal (GBS)-specific primers, (ii) a CAMP-based Group B Streptococcal (GB S)-specific probe, (iii) a second pair of Sip-based GBS-specific primers, and (iv) a Sip-based GBS-specific probe, under conditions wherein GBS-related nucleic acids are amplified; and
(b) detecting the amplified product.

9. The method of claim 8, wherein the second pair of Sip-based GB S-specific primers are the nucleic acid sequences of SEQ ID NOs:4 and 5.

10. The method of claim 8, wherein step (a) is conducted in the presence of labeled probes comprising SEQ ID NOs:3 and 6.

11. The method of claim 8, wherein the nucleic acid sample is a biological sample obtained from a patient to be tested for the presence of GBS.

12. The method of claim 8, wherein step (a) in conducted in a volume of between 0.2-100 μl.

13. An in vitro method for detecting the presence of Streptococcus agalactiae in a biological sample, comprising:
(a) releasing nucleic acids from said biological sample;
(b) performing PCR in a total volume of between 0.2-100 μl in the presence of a first pair of primers comprising SEQ ID NOs:1 and 2, and a second pair of primers comprising SEQ ID NOs:4 and 5, and labeled probes comprising SEQ ID NOs:3 and 6, under conditions wherein the presence of a Streptococcus agalactiae-related nucleic acid sequence results in an amplified and labeled PCR product; and
(c) detecting the presence of a labeled PCR product.

14. A method detecting a Group B Streptococcal (GBS) infection in a patient, comprising:
(a) obtaining a biological sample from the patient;
(b) releasing nucleic acids from said biological sample;
(c) performing PCR in a total volume of between 0.2-100 μl in the presence of a first pair of primers comprising SEQ ID NOs:1 and 2, and a second pair of primers comprising SEQ ID NOs:4 and 5, and labeled probes comprising SEQ ID NOs:3 and 6, under conditions wherein the presence of a Streptococcus agalactiae-related nucleic acid sequence results in an amplified and labeled PCR product; and
(d) detecting the presence of a labeled PCR product, wherein the presence of a labeled PCR product indicates the presence of a GBS infection.
